(19) United States
(12) Patent Application Publication
(10) Pub. No.: US 2021/0148796 A1
(43) Pub. Date: May 20, 2021

(54) CASSETTE FOR INSERTING A TISSUE SAMPLE

(71) Applicant: Thomas Märsch, Munchen (DE)

(72) Inventor: Thomas Märsch, Munchen (DE)

(21) Appl. No.: 16/641,042

(22) PCT Filed: Aug. 20, 2018
(86) PCT No.: PCT/EP2018/072417

§ 371 Date: Feb. 21, 2020

(30) Foreign Application Priority Data
Aug. 22, 2017 (DE) 10 2017 119 140.2

(51) Int. Cl. G01N 1/36; A61B 10/00

(52) U.S. Cl. CPC G01N 1/36; A61B 10/0096

(57) ABSTRACT

The invention relates to a cassette (5) for inserting a tissue sample (1), comprising an upper part (17) with a first chamber (15) and a lower part (2) with a second chamber (16) into which the tissue sample (1) is inserted. The cassette (5) is characterised in that the first chamber (15) and the second chamber (16) are spatially separated by an intermediate base (3), wherein the intermediate base (3) has at least one opening (18) via which a fluid exchange can be carried out between the two chambers (15, 16), and the cassette (5) is designed as a tightly sealed container and has a lid (4), by means of which the cassette can be sealingly closed.

The invention relates to a cassette for inserting a tissue sample according to the preamble of claim 1.

### BACKGROUND OF THE INVENTION

If tissue or cell diseases are suspected in a patient, a tissue sample is usually taken by means of a biopsy. For the subsequent microscopic examination, the tissue sample is then pre-treated chemically, embedded in a wax block, and then cut into thin slices with the aid of a microtome. The individual slices of the tissue sample may be coloured in order to make certain cell types visible. The coloured tissue slices are lastly examined for any anomalies or diseases with the aid of a microscope.

In order to produce a tissue wax block, currently the following method is usually performed: a tissue sample is firstly taken from the patient by a doctor. The tissue sample is then placed in a sample container filled with a fixing solution, and the sample container is closed and then sent to a laboratory. There, the tissue sample is removed from the sample container and inserted into a cassette. The cassette with the tissue sample contained therein is then introduced into a tissue processor (for example by the companies Sakura, Milestone, Leica), in which the tissue sample is treated with various chemicals for dehydrating the sample. Following the dehydration, the tissue sample is removed from the cassette and is transferred into a mould, which is then filled with hot, liquid paraffin. Once the wax has cooled, the finished wax block with the tissue sample contained therein is removed from the mould and cut into thin slices with the aid of a microtome, with the slices lastly being examined under a microscope.

Methods, however, for producing a tissue wax block in which the tissue sample remains in a biopsy holder, which is part of the cassette, after the dehydration are also known from the prior art. In this case the tissue sample is not removed, and instead the biopsy holder inclusive of the tissue sample contained therein is encased with hot, liquid wax. Once the wax has cooled, the biopsy holder with the finished wax block is removed and is cut into thin slices with the aid of a microtome.

All methods known from the prior art for producing a tissue wax block have the disadvantage that the tissue sample must be transferred at least once (specifically in the laboratory from the shipment container into a cassette or later also from the cassette into a mould). As a result of the current method, there are thus various ways in which the tissue sample may be contaminated or even confused with sample material from other patients. For example, contamination may occur by contaminated tweezers if the tissue sample is transferred from the shipment container (in which the doctor sent the tissue sample to a laboratory) to a cassette, or if the tissue sample is transferred from the cassette into a mould. Contaminations of the tissue sample in the tissue processor may also occur due to a poorly cleaned mould or due to contaminated work surfaces. A further disadvantage is that the medical technical assistant might breathe in poisonous fixing agent (formalin or the like) when transferring the sample into the cassette.

### OBJECT OF THE PRESENT INVENTION

An object of the present invention is therefore to create a cassette for inserting a tissue sample, with the aid of which cassette the method for producing a tissue wax block can be significantly simplified and a cross-contamination with the sample material of other patients can be prevented.

This object is solved in accordance with the invention by the features described in the main claim. Further embodiments of the invention indicated in the dependent claims.

In accordance with the invention a cassette for inserting a tissue sample is proposed, which cassette comprises an upper part with a first chamber and a lower part with a second chamber into which the tissue sample is inserted. The cassette according to the invention also contains an intermediate base, which spatially separates the first chamber and the second chamber from one another, wherein the intermediate base has at least one opening via which a fluid exchange can be carried out between the two chambers. The cassette is designed as a whole as a tightly sealed vessel and comprises a lid, by means of which the cassette can be sealingly closed. Such a cassette has the primary advantage that it can be used as a shipment container for shipping a tissue sample from the location of the biopsy to the laboratory, and as a cassette for a tissue processor, and also as a mould for producing the tissue-wax block. The entire process of producing a tissue-wax block, starting from the dispatch of the tissue sample to a laboratory as far as the encasement of the tissue sample with hot wax, can now be performed with the same cassette, without the need to transfer the tissue sample.

The lower part with the second chamber for the tissue sample preferably simultaneously forms a base part of the cassette. The base part is open towards the upper part and, besides this opening, preferably does not have any further openings through which a fluid could enter the cassette or escape from the cassette—at least not any openings that cannot be sealed closed.

The cover merely has to be removed from the cassette for the dehydration of the tissue sample in the tissue processor. The tissue sample can then be treated with various chemicals for dehydrating the sample.

In accordance with a preferred embodiment of the invention the cover is embodied in the form of a screw-on cover to close the cassette. The cassette and the cover in this case each have a corresponding screw thread. Alternatively, a cover with a detent closure or any other type of closure mechanism could also be provided.

In accordance with one embodiment of the invention the cassette may be formed such that the cover is mounted on the upper part of the cassette. To this end, the upper part may for example have a screw thread or another kind of closure mechanism.

The cassette, however, may also be designed such that the cover is mounted on the lower part of the cassette. In this case the lower part may have a screw thread or another kind of closure mechanism.

In a specific embodiment of the invention the intermediate base may be formed as a cover by means of which the second chamber can be opened or closed. The intermediate base of the opening that can be closed thereby is in this case preferably dimensioned such that a tissue sample can be inserted into the lower part from above, through the first chamber.

The intermediate base may be formed for example as a hinged cover or also as a screw-on cover. A detent mechanism may also be provided optionally, in order to fix the intermediate base in the cassette.

The upper part and the lower part of the cassette can be formed in principle either in one part or a number of parts, i.e. as separate parts.

In accordance with a first embodiment of the invention, the upper part and the lower part of the cassette are formed in one part.

In this case the intermediate base is preferably a separate part by which the second chamber can be opened or closed.

The intermediate base may be mounted in the cassette for example in articulated fashion. Alternatively, however, a detent connection or a screwed connection could also be provided.

In accordance with a specific embodiment of the invention the cassette comprises a second opening, provided in the lower part, and a second cover by means of which the second opening cannot be closed sealingly. In order to insert a tissue sample into the lower part, the second cover is opened, the tissue sample is inserted, and the second cover can then be closed again. The intermediate base in this case may also be an integral element of the cassette, which element is fixedly arranged in the cassette.

In accordance with a further embodiment of the invention the cassette comprises a separation device, with the aid of which the lower part can be separated, preferably manually,—i.e. without any tools—at least partially or completely from the upper part so as to expose the tissue-wax block. The tissue-wax block generally adheres to the underside of the intermediate base once the wax has solidified. Once the lower part has been separated, the tissue-wax block may then be further processed by clamping the upper part of the cassette with the tissue-wax block adhering thereto for example in a microtome and then cutting the block into thin slices.

The above-mentioned separation device preferably comprises a mechanically weakened region in the material of the cassette, for example one or more circumferential notches, a perforation, or another predetermined breaking point. The separation device may also comprise a tear-off lug in order to facilitate the separation of the lower part.

In accordance with a further embodiment the upper part and the lower part of the cassette are formed integrally in the form of a rough with a sealed base. The cover is dimensioned here such that it engages with an outer circumference of the trough-shaped element. The intermediate base is in this case preferably a separate part that is inserted into the trough from above through an opening. Detent means are preferably provided on an inner wall of the trough-shaped element, with the intermediate base latching with said detent means. The detent connection is preferably releasable, such that the wax block, once produced, can be removed together with the intermediate base.

In accordance with a second embodiment of the invention the upper part and the lower part of the cassette are formed as separate parts. In this case the lower part is preferably fastened releasably to the upper part. In order to couple the two parts, any connection mechanism known from the prior art can be used in principle. In accordance with one embodiment of the invention a screw connection for example can be provided. Alternatively, a detent connection or a clamped connection could also be provided.

In the case of the above-mentioned multi-part embodiment, the intermediate base is preferably an integral element which is formed in one piece on the upper part or the lower part of the cassette.

In accordance with a specific embodiment of the invention at least the lower part of the cassette is produced from a transparent material, such as clear PE or PP. A laboratory worker can thus check whether the tissue sample is situated correctly in the lower part or whether it might have shifted.

In accordance with a specific embodiment of the invention the second chamber contains a fixing element by means of which the tissue sample is mechanically fixed in the chamber. The fixing element preferably consists of an elastic material and can be produced for example from a plastic. In order to fix the tissue sample in the second chamber, a sponge for example can be secured to the underside of the intermediate base, or another fixing element, such as a protrusion, can be provided.

### BRIEF DESCRIPTION OF THE DRAWINGS

The invention will be explained in greater detail hereinafter with reference to the accompanying drawings, in which:

FIG. 1 shows a side view of a cassette formed in one part with a tissue sample inserted therein with opened intermediate base;

FIG. 2 shows a side view of the cassette from FIG. 1 with closed intermediate base;

FIG. 3 shows a side view of the cassette from FIG. 1 which has just been filled with a fixing solution;

FIG. 5 shows a side view of the cassette from FIG. 1 which has been turned upside-down in order to remove the liquid contained therein;

FIG. 6 shows a side view of the cassette from FIG. 1 with a tissue-wax block contained therein;

FIG. 7 shows a side view of the cassette from FIG. 1 which is clamped in a microtome in order to cut off a base of the cassette;

FIGS. 8a and 8b show a plan view of the cassette from FIG. 1, wherein the cassette is shown in FIG. 8a with an opened intermediate base and in FIG. 8b with closed intermediate base;

FIG. 9 shows a plan view of a one-part cassette with a tissue sample inserted therein in accordance with a second embodiment of the invention;

FIG. 10 shows a side view of the cassette from FIG. 9;

FIG. 11 shows a side view of a one-part cassette in accordance with a third embodiment of the invention;

FIG. 12 shows a perspective view of a cassette in accordance with a fourth embodiment in which the upper part and the lower part are embodied as separate parts;

FIG. 13 shows a side view of a cassette with cover in accordance with a fifth embodiment of the invention;

FIG. 14a,b each show a plan view of the cassette from FIG. 13, once in a round design and once in an angular design, in each case with inserted tissue sample;

FIG. 15 shows a side view of the cassette from FIG. 13 with inserted tissue sample, sponge, and additionally, above, an insertable intermediate base; and

FIG. 16 shows a side view of the cassette from FIG. 15 with latched intermediate base and filled fixing liquid, and closed cover.

### EMBODIMENTS OF THE INVENTION

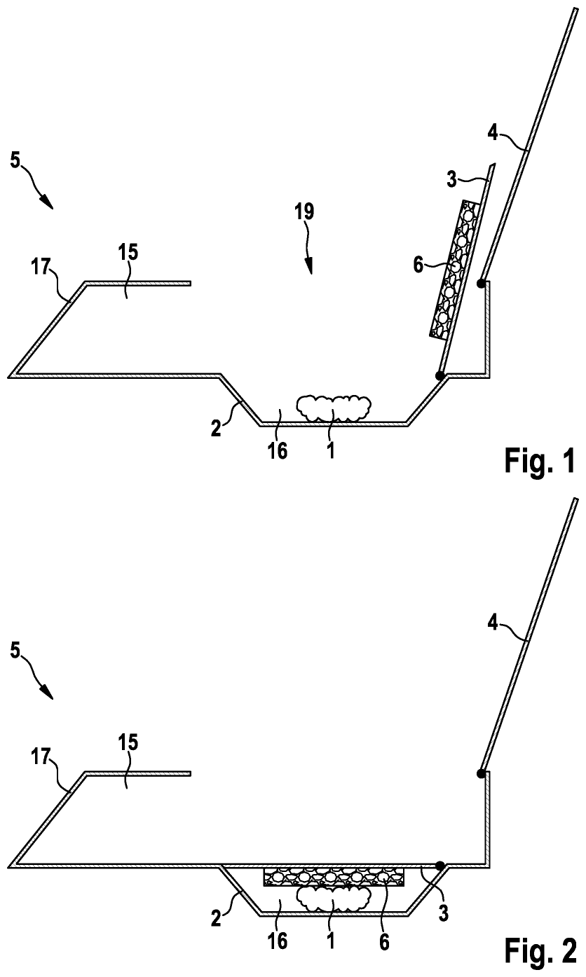

FIG. 1 shows a side view of a cassette 5 with a tissue sample 1 inserted therein. The cassette 5 comprises an upper part 17 with a first chamber 15 and a lower part 2 with a second chamber 16. The lower part 2 has a trough-shaped indentation in which the tissue sample 1 is arranged. In the exemplary embodiment of FIG. 1 the cassette 5 is formed in one part, i.e. upper part 17 and lower part 2 form an integral unit.

The cassette 5 also comprises an intermediate base 3, by means of which the first chamber 15 and the second chamber 16 can be spatially separated from one another. The intermediate base 3 in this exemplary embodiment is embodied as a hinged cover, which is mounted in the cassette 5 in articulated fashion.

The cassette 5 shown in FIG. 1 also comprises an outer cover 4, by means of which an opening 19 provided in the upper part 17 can be closed fluid-tight. In the embodiment shown here the cover 4 is formed likewise as a hinged cover which is mounted on the cassette 5 in articulated fashion. The entire cassette 5 thus forms a tightly sealed contained, which can also be used as a shipment container in order to send the tissue sample 1 from the location of removal (generally a doctor's surgery) to a laboratory, where the tissue sample is then further processed and examined.

A sponge 6 is fastened to the underside of the intermediate base 3, by means of which sponge the tissue sample 1 can be fixed when the intermediate base is closed. Alternatively a gel pad or the like could also be provided. If the intermediate base 3 is closed, the sponge 6 presses against the tissue sample 1 located in the second chamber 16 and fixes said sample in its position.

The lower part 2 of the cassette 5 consists preferably of a cuttable, chemical-resistant, transparent material, for example PE or PP.

FIG. 2 shows a side view of the cassette 5 from FIG. 1 with closed intermediate base 3. In order to hold the intermediate base 3 in the closed state, a detent means is provided here. Alternatively, the intermediate base could also be implemented for example as a screw-on cover or as a cover that can be pressed in and for example is inserted into the cassette 5 from above and latches in place as a result of pressure.

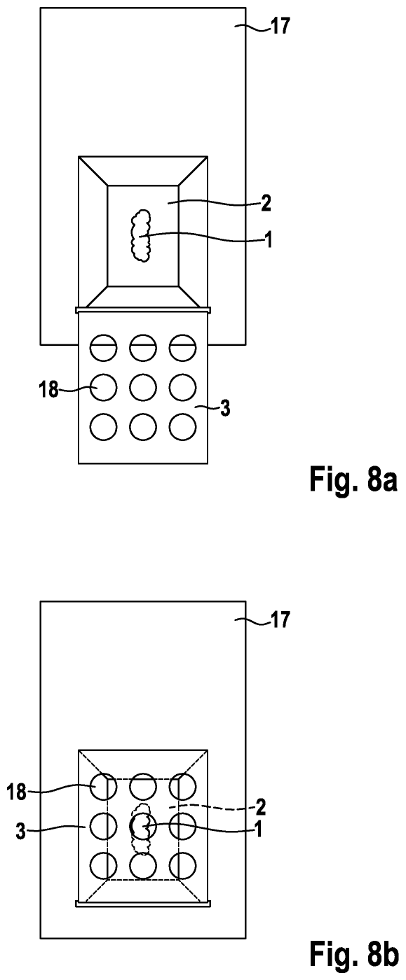

The intermediate base 3 comprises a plurality of openings 18, as is shown for example in FIGS. 8a and 8b. The openings 18 are used to enable a fluid exchange between the first chamber 15 and the second chamber 16.

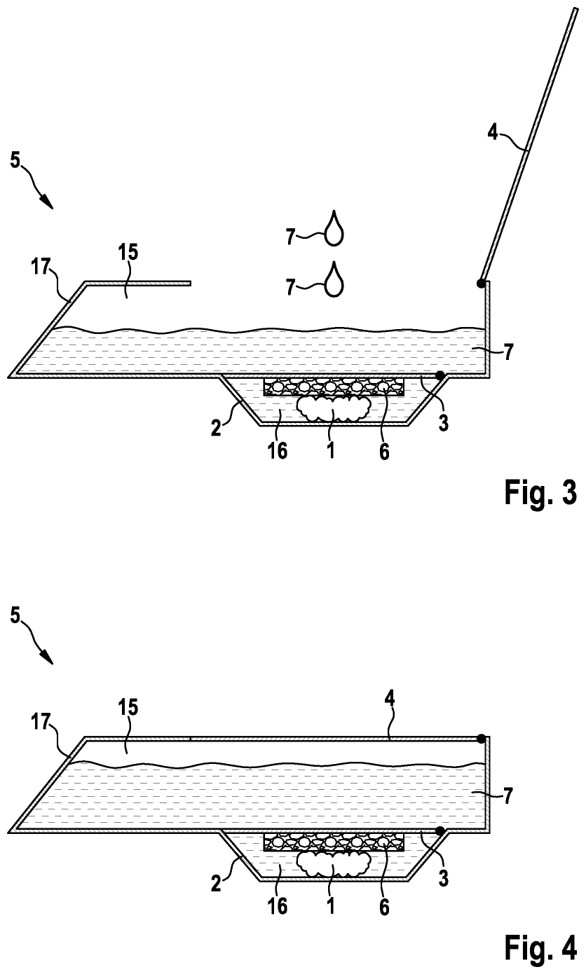

FIG. 3 shows a side view of the cassette 5 from FIG. 1 with closed intermediate base 3, which has just been filled with a fixing solution 7, by means of which the tissue sample is preserved for shipping to the laboratory.

Once the fixing solution 7 has been filled, the cover 4 is sealingly closed, as is shown in FIG. 4.

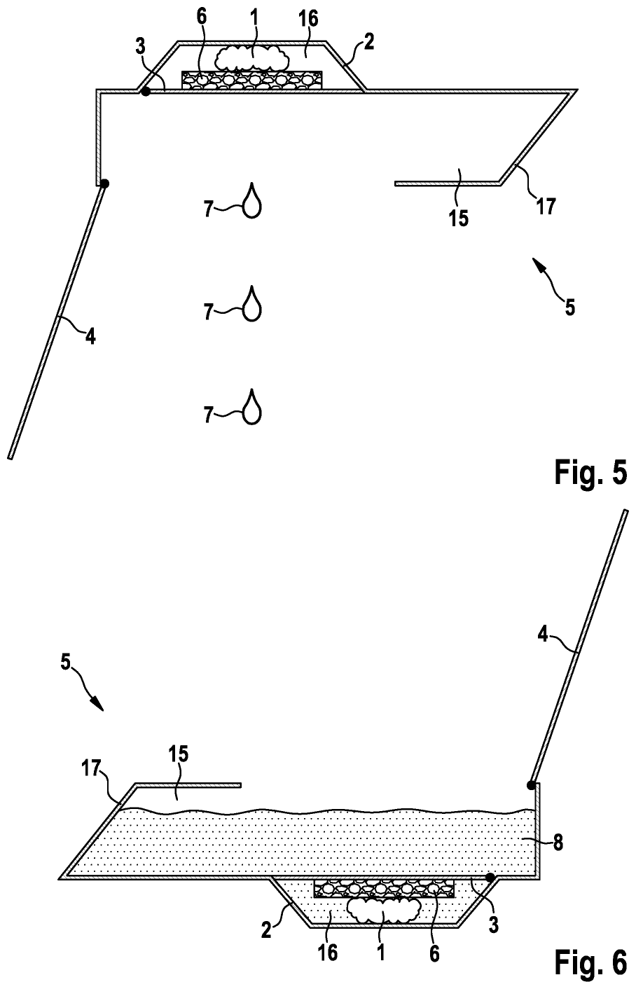

FIG. 5 shows a side view of the cassette 5 from FIG. 1 with removed cover 4, wherein the cassette 5 has been turned upside-down in order to remove a liquid contained therein.

FIG. 6 shows a side view of the cassette 5 from FIG. 1 with tissue-wax block 8 contained therein. Since the lower part 2 is made of a transparent material, it is possible to assess visually in what position the tissue sample 1 is located.

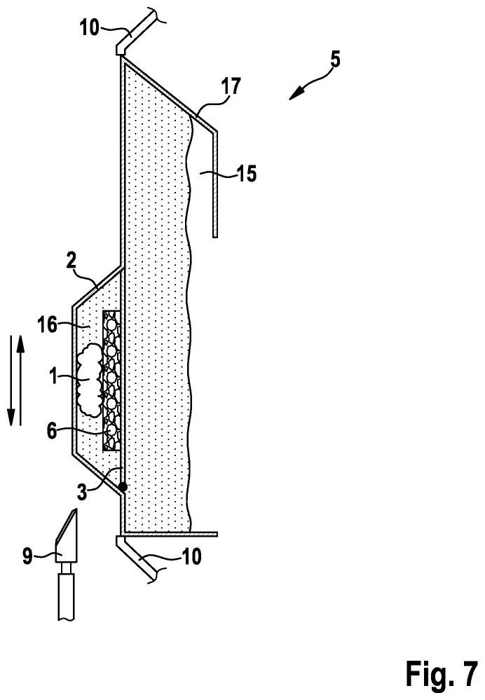

In order to lastly cut the tissue sample 1 into thin slices, the entire cassette 5 is clamped in a next step in a microtome, as is shown in FIG. 7. The holder of the microtome is denoted here by reference numeral 10. Once the cassette 5 has been clamped, the cuttable base of the lower part 2 is firstly cut away. The tissue sample 1 is then cut into thin slices, which are then examined further.

FIGS. 8a and 8b show the cassette from FIG. 1 from above, wherein the intermediate base 3 is opened in FIG. 8a and closed in FIG. 8b. As can be seen, a plurality of openings 18 are provided in the intermediate base 3, through which openings a liquid exchange may take place between the two chambers 15, 16. Instead of the round openings, the intermediate base may also be formed for example as a grid.

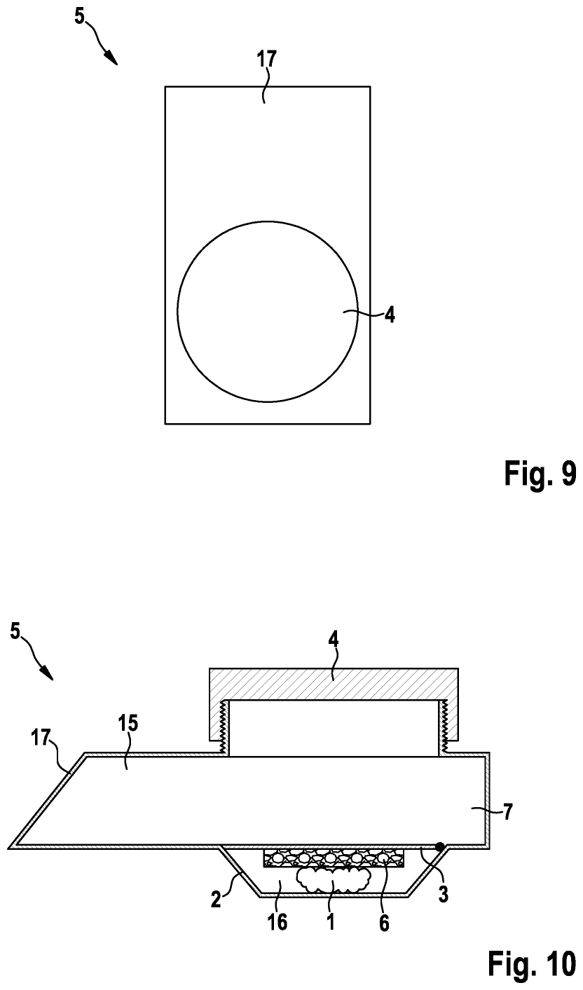

FIG. 9 shows a view of a cassette 5 in accordance with a second embodiment of the invention with a screw-on lid 4. A screw closure may be produced particularly easily and economically and enables a reliable closure of the cassette 5.

FIG. 10 again shows a side view of the cassette 5 from FIG. 9 with the screw-on cover 4.

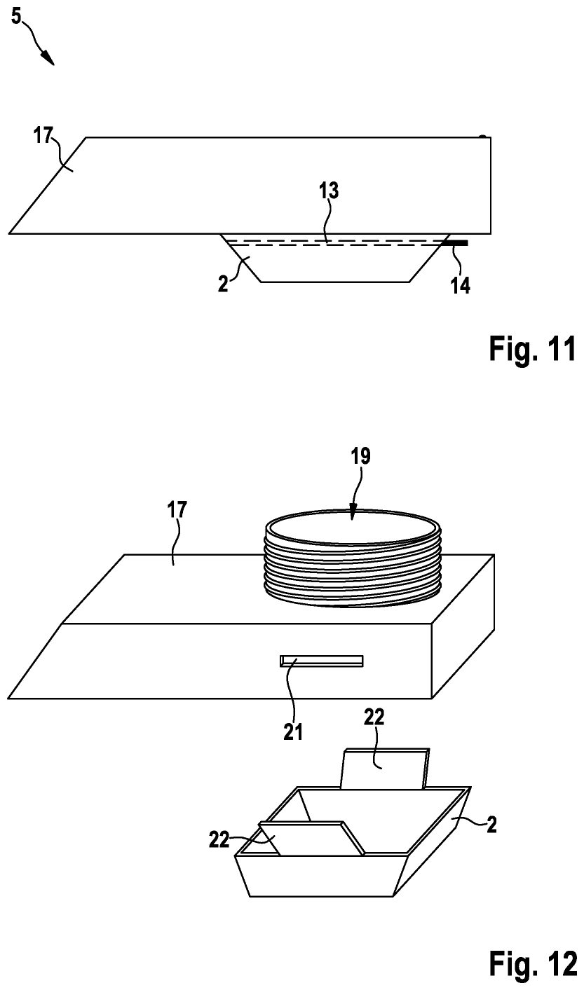

FIG. 11 shows a side view of a cassette 5 in accordance with a third embodiment of the invention. In this embodiment the cassette 5 comprises a separation device 20, with the aid of which the lower part 2 can be separated in part from the upper part 17. The separation device 20 here comprises a mechanically weakened region 13, for example one or more notches or perforations, and a tear-off tab 14, which facilitates the manual separation of the lower part 2 from the upper part 17. A separation device 20 of this kind or similar may be provided in principle on each cassette 5 according to the invention.

FIG. 12 lastly shows a perspective view of a cassette 5 in accordance with a fourth embodiment of the invention, in which the upper part 17 and the lower part 2 are embodied as separate parts. The lower part 2 is configured here in the form of a trough and comprises two lateral flanks 22, which are arranged so as to be pivotable upwardly and downwardly. In order to fasten the lower part 2 to the upper part 17, the flanks 22 are folded upwardly and latched to the upper part 17 by a detent means 21. A seal is preferably provided on the upper part 17 and lower part 2 in order to seal the assembled cassette 5. The intermediate base 3 may in turn be configured as an integral constituent of the upper part 17 or as a separate component. The lower part may also be implemented as a part that can be screwed on, having a thread.

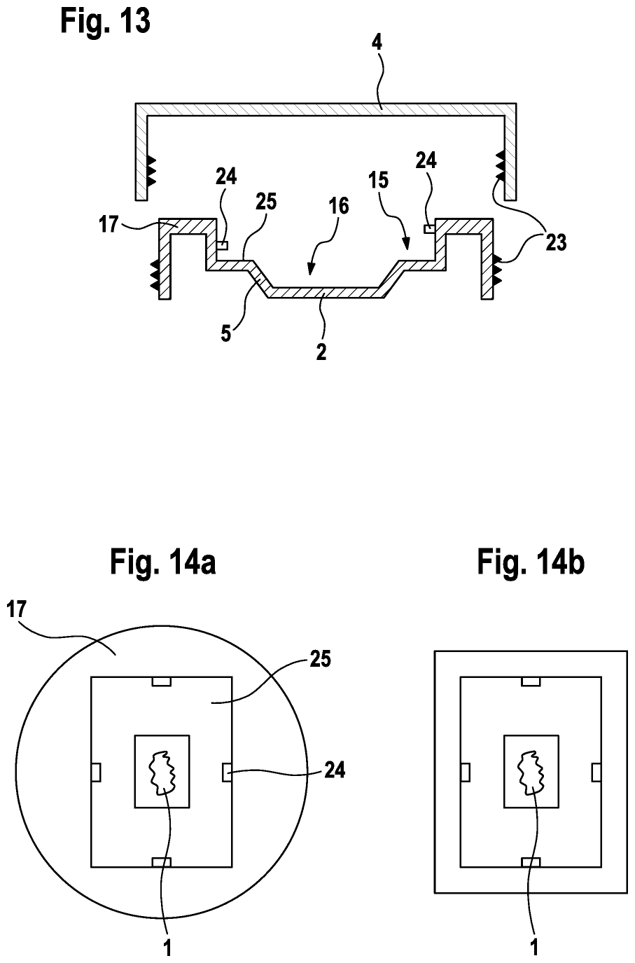

FIG. 13 lastly shows a side view of a cassette 5 according to a fifth embodiment of the invention, in which the upper part 17 and the lower part 2 of the cassette 5 are configured in one piece in the form of a trough. The cover 4 is dimensioned such that it engages with an outer circumference of the trough-like element. In the shown exemplary embodiment a screw-on closure is provided. Once the cover 4 has been screwed on, the cassette 5 is sealingly closed. Alternatively, other closing mechanisms could of course also be provided.

There is a step situated in the interior of the cassette 5, which step divides the interior into a first chamber 15 and a second chamber 16. The first chamber 15 has a greater cross-section than the second chamber 16.

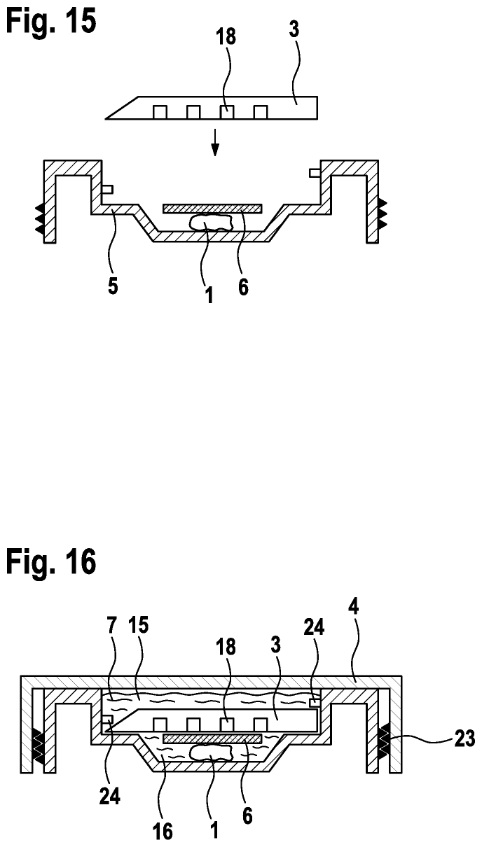

At an upper portion of the above-mentioned step there is an area 25 which is used as a support area for an intermediate base 3, which is inserted into the cassette 5 from above (see FIG. 15 or FIG. 16). In the inserted state the intermediate base 3 lies on the area 25 and is held securely in position by detent elements 24, which are formed on the inner wall of the first chamber 15. This detent connection is preferably releasable, such that the wax block, once produced, can be removed together with the intermediate base 3.

FIG. 14a and FIG. 14b show two different versions of the cassette 5 from FIG. 13. In the version of FIG. 14a the trough-like element is round. In FIG. 14b it is rectangular. Other geometries are also possible.

FIG. 15 shows the cassette 5 of FIG. 13, into which a tissue sample 1 has been inserted in the lower, second chamber 16. A sponge 6 or the like is situated above the tissue sample 1, in order to later hold the tissue sample 1 in position.

Furthermore, an intermediate base 3 with a plurality of openings 18 can be seen. Once the intermediate base 3 has been inserted, as is shown in FIG. 16, it holds the tissue sample 1 and the sponge 6 in the lower chamber 16 and divides the entire interior into the upper chamber 15 and the lower chamber 16.

For example, the base part of a currently conventional, standard tissue cassette may be used as intermediate base 3. The advantage of such a base part lies, inter alia, in that it is already obtainable in many colours and fits into existing cassette printers. Furthermore, such base parts also fit into the existing holders of microtomes, by means of which the produced wax blocks are cut

FIG. 16 lastly also shows a side view of the cassette from FIG. 15 with latched-in intermediate base 3, into which a fixing solution 7 has been filled. The cover 4 is closed by the thread 23. A seal is provided between the cover 4 and the lower part of the cassette 5 in order to seal the cassette 5 as a whole, and in particular in order to prevent an escape of liquid.

1-14. (canceled)

15. A cassette for inserting and dehydrating a tissue sample and for producing a tissue-wax block, the cassette comprising an upper part with a first chamber and a lower part with a second chamber, into which the tissue sample is inserted, wherein the first chamber and the second chamber are spatially separated by an intermediate base which has at least one opening, wherein the intermediate base has at least one opening via which a fluid exchange can take place between the two chambers, and wherein the lower part is trough-shaped and is designed to produce the tissue-wax block therein, wherein the cassette is designed as fluid-tight container and has a lid that enable the cassette to be closed fluid-tight.

16. The cassette according to claim 15, wherein the cover is a screw-on cover.

17. The cassette according to claim 15, wherein the intermediate base is formed as a cover.

18. The cassette according to claim 15, wherein the upper part and the lower part are formed in one part.

19. The cassette according to claim 15, further comprising a separation device), with the aid of which, the lower part can be manually separated from the upper part.

20. The cassette according to claim 19, wherein the separation device comprises a mechanically weakened region.

21. The cassette according to claim 19, wherein the separation device comprises a tear-off lug.

22. The cassette according to 15, wherein the upper part and the lower part are formed as separate parts.

23. The cassette according to claim 22, wherein the lower part is fastened releasably to the upper part.

24. The cassette according to claim 22, wherein the upper part and the lower part each have corresponding fastening features, with the aid of which the lower part can be fastened to or released from the upper part.

25. The cassette according to claim 15, wherein at least the lower part of the cassette is produced from a transparent material.

25. The cassette according to claim 15, wherein at least the lower part of the cassette is produced from a cuttable material.

26. The cassette according to claim 15, further including an opening defined in an upper surface thereof, the opening dimensioned such that the intermediate base can be inserted into the cassette from above.

27. The cassette according to claim 16, wherein the cover is fastened to the lower part of the cassette.
